(19) United States
(12) Patent Application Publication
(10) Pub. No.: US 2025/0195545 A1
(43) Pub. Date: Jun. 19, 2025

(54) CONCOMITANT ADMINISTRATION OF OILS FOR MANAGING NEURODEGENERATIVE DISEASES AND DEVELOPMENTAL DISORDERS

(71) Applicant: UNIVERSIDAD DE CUENCA, Cuenca (EC)

(72) Inventors: Adriana Monserrath ORELLANA PAUCAR, Cuenca (EC); Enrique Santiago PERALTA PINTADO, Cuenca (EC)

(21) Appl. No.: 18/544,023

(22) Filed: Dec. 18, 2023

(51) Int. Cl. A61K 31/00; A61K 36/47; A61K 36/9066; A61P 25/28

(52) U.S. Cl. CPC A61K 31/658; A61K 36/47; A61K 36/9066; A61P 25/28

(57) ABSTRACT

The present invention relates to concomitantly administering an effective amount of turmeric oil, sacha inchi oil, and cannabidiol in the treatment of neurological signs and symptoms in neurodegenerative diseases and developmental disorders. The neurological signs and symptoms include speech, language and communication disorders, social skills impairment, metacognition impairment, executive functions, and visual fixation patterns impairment, and pyramidal and extrapyramidal disorders associated with damage of the first or second motor neurons.

### FIELD OF THE INVENTION

The present invention relates to managing neurodegenerative diseases and developmental disorders with turmeric oil, sacha inchi oil, and cannabidiol. The neurological signs and symptoms include speech, language, and communication disorders, social skills impairment, metacognition impairment, executive functions and visual fixation patterns impairment, and pyramidal and extrapyramidal disorders associated with damage of the first or second motor neurons.

### BACKGROUND OF THE INVENTION

Communication disorder is an impairment in the ability to receive, send, process, and comprehend concepts or verbal, non-verbal, and graphic symbol systems. A communication disorder may be evident in the processes of hearing, language, and/or speech. A communication disorder may range in severity from mild to profound. It may be developmental or acquired. Individuals may demonstrate one or any combination of communication disorders (American Speech-Language-Hearing Association, 1993).

A communication disorder may result in a primary disability, or it may be secondary to other disabilities. A speech disorder is an impairment of speech sounds, fluency, and/or voice articulation. An articulation disorder is the atypical production of speech sounds characterized by substitutions, omissions, additions, or distortions that may interfere with intelligibility. A fluency disorder is an interruption in the flow of speaking characterized by atypical rate, rhythm, and repetitions in sounds, syllables, words, and phrases. This may be accompanied by excessive tension, struggle behavior, and secondary mannerisms (American Speech-Language-Hearing Association, 1993). A voice disorder is characterized by the abnormal production and/or absence of vocal quality, pitch, loudness, resonance, and/or duration, which is inappropriate for an individual's age and/or sex.

A language disorder is an impaired comprehension and/or use of spoken, written, and/or other symbol systems. The disorder may involve (1) the form of language (phonology, morphology, syntax), (2) the content of language (semantics), and/or (3) the function of language in communication (pragmatics) in any combination. Form of Language: Phonology is the sound system of a language and the rules that govern the sound combinations. Morphology is the system that governs the structure of words and the construction of word forms. Syntax is the system governing the order and combination of words to form sentences, and the relationships among the elements within a sentence. Content of Language: Semantics is the system that governs the meanings of words and sentences. Function of Language: Pragmatics is the system that combines the above language components in functional and socially appropriate communication (American Speech-Language-Hearing Association, 1993).

Social skills impairment is defined as the difficulty in social interactions related to impaired characteristics such as eye contact, smiling, appropriate facial expressions, and body postures. It is characterized by difficulty forming peer relationships and friendships (HPO, 2023).

Metacognition is the awareness of one's mental functioning and was developed in the 1970s (Flavell 1979). It is an alternative way to understand some aspects of the mental functioning of people with learning disabilities. It does not refer directly to cognitive potential but to the ability to manage it (Flavell and Wellman 1977, Brown et al. 1987). Efklides and Misaimidi (2010) described three metacognitive components: knowledge, ability, and experiences. Metacognitive experiences refer to awareness and feelings when encountering a task and processing information that is specific to it (Efklides 2008). In other words, feelings and judgments are made about a task during learning, remembering, and/or reasoning (Flavell 1979, Efklides 2001). This is the affective side of metacognition (Efklides et al. 2006). When individuals have to solve a problem, various past experiences affect their judgments relating to their ability to solve it, based on their feeling of familiarity (FOF), of difficulty (FOD), the estimate of the effort required (EOE), the estimate of solution correctness (EOSC), the interest (I) and the liking of the problem (Pleasure), the feeling of confidence in the solution produced (FOC), and the feeling of satisfaction from it (FOS). These metacognitive experiences could come to mind before, during, or after solving the problem (Hauser & Allen, 2017).

Executive functions represent a constellation of cognitive abilities that drive goal-oriented behavior and are critical to the ability to adapt to an ever-changing world. Executive functions can be split into four distinct components: working memory, inhibition, set shifting, and fluency. These components may be differentially affected in individual patients and act together to guide higher-order cognitive constructs such as planning and organization. Specific bedside and neuropsychological tests can be applied to evaluate components of executive function. While dysexecutive syndromes were first described in patients with frontal lesions, intact executive functioning relies on distributed neural networks, including the prefrontal cortex, the parietal cortex, basal ganglia, thalamus, and cerebellum. Executive dysfunction arises from injury to any of these regions, their white matter connections, or neurotransmitter systems. Dysexecutive symptoms, therefore, occur in most neurodegenerative diseases and many other neurologic, psychiatric, and systemic illnesses (Rabinovici GD, 2015).

Pyramidal and extrapyramidal disorders are associated with first or second motor neuron damage. The pyramidal system is described as being involved with the initiation and control of “voluntary” movements, whereas the extrapyramidal system controls involuntary movements (Sengul and Watson, 2014). Extrapyramidal motor disease, therefore, is generally characterized by impaired motor control, usually resulting in basal ganglionic dysfunction (Sanders and Gillig, 2012). The World Health Organization's International Classification of Diseases includes a classification for extrapyramidal and movement disorders (Jankovic, 1995). This World Health Organization category includes, among others, Parkinson's disease, secondary parkinsonism, other degenerative diseases of the basal ganglia, and clinical syndromes that result in dystonia, dyskinesia, essential tremor, and other forms of tremor and chorea (Dorman, 2015).

Turmeric (Curcuma longa L.) belongs to the family Zingiberacea and is native to southeast India. This old perennial plant that Asians have used for thousands of years is a major part of the Siddha system. This system has recommended turmeric for medicine. Curcuminoids and essential oil from turmeric have shown various bioactivities and promising results in various research investigations. Hence, the oil has been in high demand since ancient times and, recently, finds extensive application in flavor, perfumery, cosmetic, food products, beverages, and the pharmaceutical industry (Kuntal, 2016).

Sacha inchi (Plukenetia volubilis) is an oleaginous plant that produces oil-and protein-rich seeds. It has been cultivated for centuries and is native to the tropical rainforest of the Amazon region of South America, including parts of Peru and northwestern Brazil. At present, sacha inchi seeds are emerging as a potential source of macro-and micronutrients, α-linolenic acid, and phytochemicals. Plukenetia volubilis seeds are identified as a potential source of PUFA-rich oil and protein. Sacha inchi seeds contain 33.4-54.7% fat, 24.20-33.30% protein, 6.59-30.90% carbohydrates, 6.61-11.30% fibers, and 2.70-6.46% ash (on a dry basis) (Bueno-Borges et al., 2018, Chirinos et al., 2013, Muangrat et al., 2018, Takeyama and Fukushima, 2013). Raw sacha inchi seeds are astringent in taste due to the presence of phytotoxins and, thus, are not edible (Ankit Goyal, 2022).

The plant Cannabis sativa, known as marijuana or cañamo is composed of more than 60 cannabinoids. The two main components of the plant are Δ⁹-tetrahydrocannabinol (THC), which is responsible for psychoactive effects, and cannabidiol (CBD), the main component of the plant that does not have psychological or behavioral effects. Other substances found in marijuana are (1) cannabinol, which has a slightly less potent effect than THC; (2) cannabigerol (CBG), a substance that is not as psychoactive as CBD; and (3) β-caryophyllene (I.M.P. Linares, 2017).

CBD and CBG are the major non-psychotropic cannabinoids found in Cannabis sativa. C. sativa is the principal cannabinoid in fiber-type Cannabis (in its carboxylic acid, CBDA), a plant easily available to researchers, in contrast to the strictly controlled drug-type Cannabis varieties. Second to THC, the pharmacological effects of CBD have been best studied of all cannabinoids. It has powerful antioxidant properties, more potent than ascorbate and α-tocopherol. Also, it has notable anti-inflammatory and immunomodulatory effects. Furthermore, sedating, hypnotic, antiepileptic, and anti-dystonic effects have been described. Also, CBD modulates some opioid receptors and can modulate sleep in rats. Moreover, CBD was found to have antianxiety effects. It was concluded that CBD possesses anxiolytic properties, possibly mediated by an action on limbic and paralimbic brain areas, where it reduced regional cerebral blood flow. These anxiolytic properties might prove useful in psychiatry (Arno Hazekamp, 2010).

U.S. Pat. No. 9,782,361 relates to the anticonvulsant activity of turmeric oil and its volatile bisabolene sesquiterpenoids ar-turmerone, α-turmerone, β-turmerone (curlone), and α-atlantone, as an anticonvulsivant agent for the treatment of epilepsy and/or as a therapeutic agent for the treatment of disorders of the central nervous system, including tremor, pain, mood disorders (including depression, bipolar disorder, attention deficit-hyperactivity disorder, and schizophrenia), and neurodegenerative diseases.

The article “Neuroprotective Effect of Sacha Inchi Oil (Plukenetia volubilis L.) in an Experimental Model of Epilepsy” describes an experimental study to determine the protective effect of sacha inchi oil on pentylenetetrazole-induced seizures in albino mice (Herrera-Calderon, Ángel, Tinco-Jayo, & Enciso-Roca, 2019).

Article “Cannabis and Turmeric as Complementary Treatments for IBD and Other Digestive Diseases” describes that the use of cannabis and turmeric is potentially beneficial in inflammatory bowel disease (IBD) and irritable bowel syndrome (IBS); however, neither has been compared to standard therapy in IBD and thus should not be recommended as an alternative treatment for IBD. For cannabis, in particular, additional investigation regarding appropriate dosing and timing, given the known adverse effects of its chronic use, and careful monitoring of potential bleeding complications with synthetic cannabinoids is imperative.

Therefore, there is a need for the research and development of alternative therapies for the treatment of signs and symptoms of neurodegenerative diseases and developmental disorders.

### BRIEF DESCRIPTION

The present invention relates to concomitantly administering an effective amount of turmeric oil, sacha inchi oil, and cannabidiol in the treatment of neurological signs and symptoms in neurodegenerative diseases and developmental disorders. The neurological signs and symptoms include speech, language and communication disorders, social skills impairment, metacognition impairment, executive functions, and visual fixation patterns impairment, and pyramidal and extrapyramidal disorders associated with damage of the first or second motor neurons.

### BRIEF DESCRIPTION OF THE FIGURES

FIG. 1. Brain resonance imaging showing hyperintensities in supratentorial and infratentorial white matter, with greater involvement in the right hemisphere before the administration of turmeric oil and cannabidiol (CBD).

FIG. 2. Brain resonance imaging after two weeks displaying severe supratentorial and infratentorial involvement.

FIG. 3A. Slice of a brain's MRI performed in March 2023 showing a significant decrease in hyperdensities, areas of gliosis, and encephalomalacia, with no progression of the disease on neuroimaging.

FIG. 3B. Slice of a brain's MRI performed in March 2023 showing a significant decrease in hyperdensities, areas of gliosis, and encephalomalacia, with no progression of the disease on neuroimaging.

FIG. 3C. Slice of a brain's MRI performed in March 2023 showing a significant decrease in hyperdensities, areas of gliosis, and encephalomalacia, with no progression of the disease on neuroimaging.

FIG. 3D. Slice of a brain's MRI performed in March 2023 showing a significant decrease in hyperdensities, areas of gliosis, and encephalomalacia, with no progression of the disease on neuroimaging.

FIG. 3E. Slice of a brain's MRI performed in March 2023 showing a significant decrease in hyperdensities, areas of gliosis, and encephalomalacia, with no progression of the disease on neuroimaging.

FIG. 3F. Slice of a brain's MRI performed in March 2023 showing a significant decrease in hyperdensities, areas of gliosis, and encephalomalacia, with no progression of the disease on neuroimaging.

### DETAILED DESCRIPTION OF THE INVENTION

Most pharmacological activities of turmeric have been explained by the properties of curcumin, mainly because turmeric oil has not been as extensively studied as curcuminoids. Turmeric rhizome oil (TO) is responsible for this spice's characteristic taste and smell. Dried rhizomes contain about 3-6% essential oil. The major TO constituents are sesquiterpenes: bisabolanes, guaianes, germacranes, caranes, elemanes, spironolactones, selinanes, santalanes, and caryophyllanes. Ar-turmerone, α-turmerone, and β-turmerone are the principal bisabolane sesquiterpenes. Other notable TO compounds with reported bioactivity are a-atlantone, ar-curcumene, γ-curcumene, curlone, p-cymene, z-citral, eucalyptol, β-(Z)-farnesene, germacrone, β-sesquiphellandrene, α-santalene, α-zingiberene, and l-zingiberene (Orellana-Paucar & Machado-Orellana, 2022).

Turmeric oil is extracted from the rhizomes and in this case the total yield is around 3-7%. Turmeric oil is extracted from ground turmeric root by steam distillation. With this method, the volatile oil released from the spice is transported along with the vapors and collected. The mixture of volatile oil and steam is then cooled in a condenser. Additionally, turmeric oil is separated from water and collected for drying and stored in a cool, dry place protected from light (Ravindran, Babu, and Sivaraman 2007). The volatile oil of turmeric is light yellow in color and is responsible for the characteristic aroma of turmeric. Its major components are mainly oxidized sesquiterpenes.

The term “essential oil,” “essential oil of turmeric,” or “turmeric oil” refers to “volatile oil,” “curcuma oil,” “volatile curcuma oil,” or “volatile oil of turmeric.” The essential oil is a mixture of oils obtained during the extraction of curcumin or curcuminoids from turmeric rhizomes. Turmeric oil is mainly constituted by turmerones, α-turmerone (30-32%), β turmerone (15-18%), and ar-turmerone (dehydroturmerone) (17-26%) (Orellana-Paucar A, 2020).

Sacha inchi (Plukenetia volubilis L.), is the oleaginous plant of the Euphorbiaceous family, originally cultivated in the Amazon Forest. It is traditionally appreciated and consumed as the healthy food. In vivo, in vitro and clinical studies have suggested the beneficial effects of sacha inchi (SI) for neuroprotection, dermatology, antidyslipidaemic, antioxidant and anti-inflammatory, antiproliferative and antitumor modulation activities. Many of these potential impacts are related to its bioactive compounds, particularly essential fatty acids, proteins, and phytochemicals (Mhd Rodzi NAR, 2022).

The term “sacha inchi essential oil” or modifications thereof refers to a vegetable oil like olive, avocado, wheat germ, rice bran, and argan oils. Sacha inchi oil is a cold-pressed oil from the seeds of Plukenetia volubilis L. The seeds contain a high proportion of proteins (25-30%) and oil (35-60%). The main constituents of the oil proportion are omega-3, omega-6, and oleic acid. Also, natural antioxidants, including tocopherols and polyphenols have been found in sacha inchi oil (Wichuda, 2020) (Sarawut, 2022).

The term “CBD oil” or their variations refer to a hemp extract containing cannabidiol (CBD). Cannabis sativa possesses a wide variety of active compounds. Among them are the cannabinoid (Legare, 2022). CBD is the dominant cannabinoid found in industrial hemp, a term that is used for cannabis varieties that have been used for fiber and seed oil production and that contain high concentration of CBD and low concentration of the main psychoactive cannabinoid, (−)-trans-Δ9-tetrahydrocannabinol (Δ9-THC or THC). A number of isomers of THC, such as (+)-cis-Δ9-THC and Δ8-THC, can be found in the cannabis plant, but they are generally present in minor amounts. These compounds are produced in the stems, leaves, and flowers of growing plants in their carboxylated form (Teresa Moreno, 2020).

As used herein, the term “disorder” or “disease” broadly refers to a syndrome, condition, chronic illness, or a particular disease. For example, neurodegenerative diseases and developmental disorders. The neurological signs and symptoms include speech, language and communication disorders, social skills impairment, metacognition impairment, executive functions, visual fixation patterns impairment, and pyramidal and extrapyramidal disorders associated with damage of the first and/or second motor neurons.

As used herein, the terms “treat,” “treated,” “treatment,” and variations thereof mean subjecting an individual subject to a protocol, regimen, processor remedy, in which it is desired to obtain a physiologic response or outcome in that subject e.g., a patient. However, because every treated subject may not respond to a particular treatment protocol, regimen, or process, treating does not require that the desired physiologic response or outcome be achieved in each subject or subject population. Accordingly, a given subject or subject population may fail to respond or respond inadequately to treatment. The term “clinical response,” as used herein, means a reduction of the severity or number of symptoms or characteristics of a disorder or disease, during or following treatment.

The term “administering,” “administration,” or variations thereof, as used herein, means introducing an agent e.g., turmeric oil into the body of a subject, such as a human, in need of such treatment. Said administration could be by different routes of administration, e.g., oral, subcutaneous, cutaneous.

In a present invention, an “effective amount” or “therapeutically effective amount” of turmeric oil, sacha inchi or CBD is an amount of such material that is sufficient to produce beneficial or desired results as described herein when administered to a subject. Effective dosage amounts will vary with the route of administration, the rate of excretion, the duration of the treatment, the identity of any other drug being administered, the age of the patient, or the patient weight. In general, a suitable dose of the turmeric oil, sacha inchi oil and CBD disclosed herein or a composition containing the same will be that amount of the active agent e.g., turmeric oil, sacha inchi oil and CBD which is the lowest dose effective to produce the desired effect. The therapeutic index of turmeric oil is based on ar-turmerone content and corresponds to 1-50 mg ar-turmerone/kg/day. For sacha inchi, oil is 0.1-0.3 mg/kg/day and, for CBD, it is 1-10 mg/kg/day.

A suitable, non-limiting example of a dosage of turmeric oil according to the present invention may be about 1 mg/kg/to about 500 mg/kg. However, doses employed for human treatment typically may be in the range of 1 mg/kg/day to 50 mg/kg/day.

With respect to the dosage of the sacha inchi oil according to the present invention, it may be about 0.1 mg/kg to about 10 mg/kg. However, doses employed for human treatment typically may be in the range of 0.1 mg/kg/day to 0.3 mg/kg/day.

A suitable, non-limiting example of a dosage of CBD according to the present invention may be about 1 mg/kg to about 500 mg/kg/day. However, doses employed for human treatment typically may be in the range of 1 mg/kg/day to 10 mg/kg/day.

As used herein, “modifying the course of treatment” refers to any change in the subject's treatment type and/or dosage, including administering different dosages to the subject, stopping, or omitting treatment.

In this embodiment, the obtaining, determining, and administering steps have been previously disclosed. As used herein, the term “ameliorate” “ameliorating,” and grammatical variations thereof mean to decrease the severity of the symptoms, particularly negative symptoms of a disease in a subject, preferably in a human.

In one aspect of this embodiment, carrying out the method results in reducing or eradicating negative symptoms, including but not limited to speech, language and communication disorders, social skills impairment, metacognition impairment, executive functions, and visual fixation patterns impairment, and pyramidal and extrapyramidal disorders associated with damage of the first or second motor neurons.

In one embodiment, the method results in improvement of language, walking abilities, and neurodevelopmental abilities, and social skills, improvement of reflexes, and psychomotor development.

### EXAMPLES

### Case 1: Mitochondrial Disease and Leigh Syndrome

Male patient, born in April 2019, of non-consanguineous parents, with no significant history from pregnancy or childbirth. The parents report that their first daughter died at one year of age due to progressive deterioration, severe hypotonia, severe neurodevelopmental delay, epilepsy, and with lesions on brain magnetic resonance imaging (MRI) that led to suspicions of leukodystrophy. The child died without a precise etiological diagnosis.

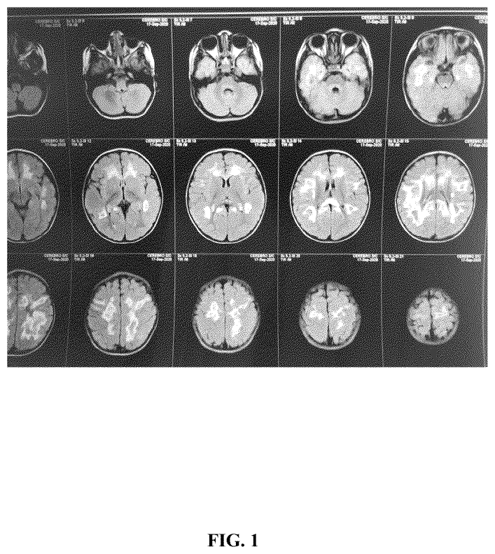

The first consultation with the patient took place at the age of 1 year and 4 months. Parents referred that, from 6 months, a left-hand preference was present in their son and, since the patient was 1 year and 3 months old, he showed progressive loss of strength towards the left side of the body, anorexia, and loss of gait with ataxia. The patient did not crawl, and showed asthenia, decreased appetite, frequent yawning, and loss of language. In the neurological examination, dyskinesias of the left hemibody stood out, and mild tetraparesis with increased reflexes was also identified. Constipation was reported as another complication. Brain tomography and resonance imaging were performed, showing hyperintensities in supratentorial and infratentorial white matter, with greater involvement in the right hemisphere (FIG. 1). It was suggested to start the administration of turmeric oil and cannabidiol (CBD).

In November 2020, two pathogenic variants identified in NDUFV1 were confirmed. NDUFV1 is associated with Autosomal Recessive Mitochondrial Complex I Deficiency.

| | | | VARIAN |
|---|---|---|---|
| GENE | VARIANT | ZYGOSITY | CLASIFICATION |
| NDUFV1 | c-1268C > T | homozygous | PATHOGENIC |
| | (p.Thr423Met) | | |
| GALT | c-119_*116del (Non- | homozygous | PATHOGENIC |
| | coding) | | |
| GCDH | c-1198G > A | homozygous | PATHOGENIC |
| | (p.Val400Met) | | |
| PEX2 | c.104 > T (p.Arg4*) | homozygous | PATHOGENIC |
| SCO1 | Deletion (Exon 5) | homozygous | Uncertain Significance |
| TSEN54 | c.1166_1167delinsCC | homozygous | Uncertain Significance |
| | (p.Gln389Pro) | | |

Between October and December 2020, improvement was evident and considerable. The yawning subsided and his language improved. The patient recovered walking with the support of two hands, although his gait remained unstable. He crawled without difficulty.

In July 2021, the parents' patient returned to consultation because they suspended the administration of turmeric oil and CBD to their son, which led to a new neurodevelopmental regression: loss of gait, loss of sitting, language, and social smile. During the examination, he appeared to understand some commands, neck control was partial, and divergent squint was evident in his eyes. In addition, axial hypotonia and peripheral hypertonia, ROT ++++/++, and bilateral plantar extensor reflex were observed. The patient showed frequent events of respiratory pauses. Two weeks later, the patient was hospitalized because he showed cardiorespiratory arrest and entered intensive care with a clinical picture compatible with Leigh Syndrome. Brain magnetic resonance imaging showed severe supratentorial and infratentorial involvement (FIG. 2).

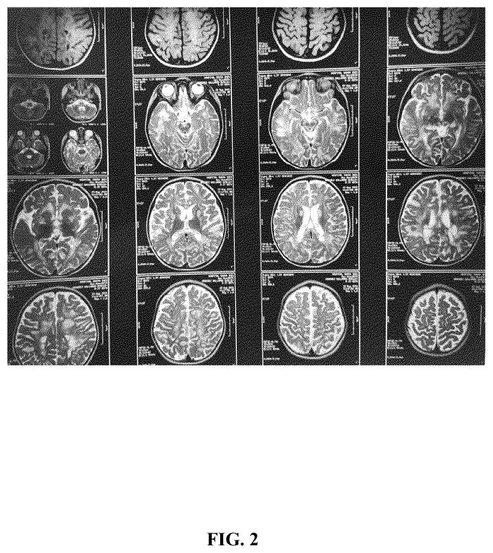

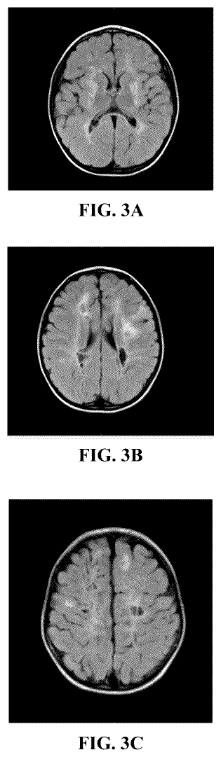

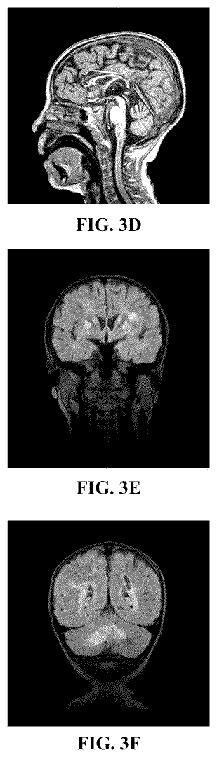

Daily treatment with 12.6 mg turmeric oil, 40 mg CBD and 3 ml sacha inchi oil was initiated. Concomitant administration of CBD, turmeric oil and sacha inchi oil continued, achieving complete recovery of his language, managing to walk with the support of one hand, partially correcting strabismus, and with improvement of axial hypotonia and peripheral hypertonia. Reflexes presented a slight increase; the Babinski reflex remained only on the left side. Brain MRI performed in March 2023 showed a significant decrease in hyperdensities, areas of gliosis, and encephalomalacia, with no disease progression on neuroimaging (FIG. 3A to FIG. 3E).

### Case 2: Charcot-Marie-Tooth Disease Type 4F (CMT4F; OMIM #614895)

Female patient born in 2015. The patient's first consultation took place at the age of 3. The patient was the daughter of non-consanguineous parents without significant prenatal or natal history. Her psychomotor development was apparently normal until the patient was 8 months old. The patient's mother referred that her daughter did not crawl and started walking at 21 months. The patient's language was according to her age. Parents came to the consultation because their daughter's walk was not normal. The neurological examination highlighted mixed hypotonia, slight retraction of the Achilles tendons, slight girdle weakness, ROT +/++, bilateral plantar flexor reflex. The Gowers sign was incomplete.

In May 2019, electromyography and nerve conduction velocity evaluation showed primarily demyelinating sensory-motor polyneuropathy without signs of denervation, suggestive of Charcot-Marie-Tooth disease. A molecular study and physical therapy were requested.

In December 2019, the patient was attending school without problems in her performance. Upon examination, a greater difficulty for walking was noted; it was not possible for her to go up or down the stairs, her strength decreased, and she presented generalized areflexia. A daily administration of 12.6 mg turmeric oil started.

In May 2020, adherence to the administration of turmeric oil was reported as irregular. Despite this, gait and strength improved. Molecular examination reported the identification of a homozygous frameshift variant in the PRX gene, previously associated with type 4F Charcot-Marie-Tooth disease (CMT4F; OMIM #614895), considered highly possibly pathogenic and responsible for the phenotype of the patient disease.

In August 2020, strength improved, and the patient had less difficulty for standing up. Thus, 20 mg CBD was added to the daily treatment.

In November 2020, the patient began to run and climb stairs independently. She stood up without difficulty.

In May 2021, the electromyography and the nerve conduction velocity analysis reported sensory-motor polyneuropathy, primarily demyelinating type, without signs of active severe degree denervation. In relation to the previous study, no significant changes were found, that is, no progression of the disease was observed. The patient ran without difficulty, went up and down the stairs without support. Thus, 5 ml of sacha inchi oil was added to the CBD and turmeric oil treatment.

In April 2023, the patient went up and down the stairs without support, ran, and jumped. She was not attending physical therapy or gymnastics. Administration of turmeric and sacha inchi oils with CBD was maintained.

### Case 3: Charcot-Marie-Tooth Disease Type 1A (CMT1A)

Female patient, born in 2006. The patient attended her first consultation at 8 years and 9 months of age. The patient was the daughter of non-consanguineous parents. She had no significant prenatal and natal history. The mother described a normal psychomotor development until her daughter was 2 and a half years old, when her parents noticed she had difficulty climbing the stairs. Since then, her motor defect slowly progressed with increasing weakness and frequent falls. Her physical examination revealed generalized weakness, abolished reflexes, preserved sensitivity, a myopathic gait, cavus feet with distal tendon retractions, and lumbar scoliosis.

At 5 years of age, after undergoing an electromyography and nerve conduction velocity study, sensory and motor neuropathic compromise was found, of a demyelinating type without signs of active denervation in the four extremities, greater and of severe degree in the lower extremities. Her muscular development was normal. Therefore, Charcot-Marie-Tooth disease was suspected, and a molecular examination was requested.

In October 2020, they started daily administration of 27 mg of turmeric oil. In December of the same year, the patient's strength and tone improved.

In May 2021, a molecular examination was obtained with the following result: “a pathogenic variant, Gain (Entire coding sequence), identified in PMP22. A copy number gain of the entire PMP22 gene is associated with autosomal dominant Charcot-Marie-Tooth disease type 1A (CMT1A) (MedGen UID: 75727). This result is consistent with a predisposition to, or diagnosis of, Charcot-Marie-Tooth disease type 1A.” Based on this result, 50 mg/day of CBD was added to the administration of turmeric oil.

In July 2022, the patient's gait improved, she went up and down stairs with difficulty, she had no falls, her gait presented minor difficulty, retractions and cavus feet were regressing, and scoliosis did not progress.

### Case 4: Communication and Language Disorder, Autism, Attention Deficit, Impaired Visual Fixation

Female patient of 2 years and 3 months of age. Daughter of non-consanguineous parents, with no significant prenatal or natal history. The parents brought her daughter to the consult because the patient did not respond to the name, understood or obeyed orders. The patient did not speak, was very restless, had no visual fixation, and presented constant fluttering. Melatonin was administered to sleep because the patient was waking up often at night.

ADI-R and ADOS-2 tests were applied, and the score greater than 24 indicated moderate to severe concern. In the neurological examination there were no sensory, motor, or cranial nerve alterations. Her hearing function was normal. The parents reported that the patient had been very irritable and verbally repeating her desire to avoid medication. Joint administration of 14.4 mg turmeric oil and CBD started. Six months after this concomitant administration, the patient displayed at least 30 words in her language, was capable of forming sentences, understood commands, and communicated her needs.

### First Evaluation

The patient appeared dressed and clean according to her age and gender. After initial play activities, she managed to join them with an exploratory game and achieved functional play. The patient could maintain her gaze for short periods, her attentional focus was sustained and showed a proper level of motor activity. The patient explored the environment; at the beginning, she was shy, but she managed to adapt. The patient did not require the mother's presence to stay in the evaluation room, and she managed to be at the work room for a period of 40 minutes, with breaks every 10 minutes.

The patient's behavior was characterized by exploring the room and the objects presented (dolls and kitchen toys). She explored the objects and managed to integrate them into a functional game.

Interactive competence: In the games, the patient showed communication with her mother, she pointed at objects that she liked and participated in the proposed activities. She was able to maintain eye contact for short periods.

Communicative aspects: When the patient wanted a certain object, if she could not get it by herself, she had difficulty asking for it.

Spontaneous verbal emissions: The patient emitted single words and short phrases with articulatory difficulties. Sometimes, she repeated words mentioned by other people.

Comprehensive language: The patient responded to her name without difficulty, understood and executed commands after modeling. In the case of verbal instructions, these were repeated several times.

During the evaluation sessions, the patient showed sporadic echolalia, and her tone of voice was flat.

### Psycho-Educational Profile

### WECHSLER Intelligence Scale for Preschool and Elementary School (WIPPSI IV)

| TEST | PD | | | | | | |
|---|---|---|---|---|---|---|---|
| Cubes | 12 | 7 | | 7 | | | |
| Information | 10 | 4 | 4 | | | | |
| Arrays | 14 | 14 | | | 14 | | |
| Animal search | 10 | 6 | | | | | 6 |
| Recognition | 10 | 7 | | | | 7 | |
| Similarities | 6 | 6 | 6 | | | | |
| Concepts | 6 | 8 | | | 8 | | |
| Cancelation | 8 | 4 | | | | | 4 |
| Location | 7 | 7 | | | | 7 | |
| Puzzle | 14 | 8 | | 8 | | | |
| Sum Point Scalars | | 44 | 10 | 15 | 22 | 14 | 10 |
| | | | VCI | VSI | FRI | WMI | PSI |

| | | |
|---|---|---|
| SCALE | Scalar Score | CI |

| | | | |
|---|---|---|---|
| Verbal Comprehension Index | VCI | 10 | 72 |
| Visuospatial Index | VSI | 15 | 85 |
| Fluid Reasoning Index | FRI | 22 | 106 |
| Work Memory Index | WMI | 14 | 81 |
| Processing Speed Index | PSI | 10 | 73 |
| FULL SCALE | OIQ | 44 | 79 |

Reference mean: 90/109

Overall intellectual quotient (OIQ) or cognitive potential: 79 low intellectual capacity, cognitive potential with needs.

Results suggested the patient had a total intelligence quotient of 79 points, equivalent to low. Thus, there was a 95% chance that her total IQ is between 74-87, with needs in verbal comprehension and processing speed. The patient displayed a significant strength in fluid reasoning.

Verbal comprehension index (VCI): (72=low). Low performance was observed in relation to the average in the measure of knowledge acquired from the environment, the formation of verbal concepts, and verbal reasoning of the patient. It is noteworthy that, in this test, the patient must give verbal answers to all the items applied.

Visuospatial Index (VSI): (85=medium low). Low average performance was observed in the measure of visuo-spatial processing, of the integration and synthesis of relations, of the attention to visual details, of the formation of non-verbal concepts and of visuomotor integration. This index requires the manipulative IQ.

Fluid reasoning index (FR): (106=medium). Average performance was observed in the patient's fluid reasoning, ability to detect underlying notional relationships between visual objects, and employed reasoning to identify and apply rules. Inductive and quantitative reasoning, broad visual intelligence, and simultaneous processing were evaluated.

Working memory index (WMI): (81=medium low). This index measures visual working memory, visuospatial working memory, and the ability to show resistance to interference. This scale is related to the evaluation of attention and concentration, mental control, and reasoning.

Processing Speed Index (PSI): (73=low). Low relative-to-average performance was observed in the ability to explore, order, or discriminate simple visual information. This scale also measures short-term visual memory, visuomotor coordination, cognitive flexibility, and test-taking speed.

Discrepancy analysis: Within the table of comparisons between the scores obtained by the patient, a strong point in fluid reasoning was observed. The patient presented a weak point in verbal comprehension and processing speed.

### Child Neuropsychological Maturity Questionnaire CUMANIN

| SCALE | PERCENTILE | EQUIVALENCE |
|---|---|---|
| PSYCHOMOTOR | 30 | MEDIUM LOW |
| ARTICULATORY | 5 | VERY LOW |
| LANGUAGE | | |
| EXPRESSIVE LANGUAGE | 15 | LOW |
| COMPREHENSIVE | 5 | VRY LOW |
| LANGUAGE | | |
| SPACE | 5 | VERY LOW |
| VISOPERCEPTION | 10 | LOW |
| ICONIC MEMORY | 25 | MID LOW |
| RHYTHM | 30 | MEDIUM |
| ATTENTION | 5 | VERY LOW |
| CD | LESS THAN 65 | VERY LOW |

From the analysis of these results, it was concluded that the patient had a global neuropsychological development quotient with a percentile of less than 65 (very low) in relation to the expected mean for her age. Thus, a delay in verbal development with significant expressive, comprehensive, and articulatory compromise was found. In addition, visual motor skills and attention deficit were identified.

### Tepsi Psychomotor Test

| | DESCRIPTIVE | EXPECTED |
|---|---|---|
| SUBTEST | CATEGORY | SCORE |
| COORDINATION | 24: delay | 40-50 |
| LANGUAGE | 39: risk | 40-50 |
| MOTOR | 36: risk | 40-50 |
| TOTAL SCORE | 31: risk | 40-50 |

The results suggested the patient was within the risk zone for delay in language development, psychomotor skills, and coordination.

### Basic Skills Assessment

The patient was able to:
Identify parts of her body.
Identify spatial notions: above, below, next to, behind, near.
Jump with her feet together, stand on one foot for 5 seconds.
Draw a straight line.
Identify the onomatopoeic sound of animals.
Identify primary and secondary colors.
Count to 10.
Identify the vowel: a, i, o, u.
Identify basic geometric figures: circle, square, triangle.
Build a 4-piece puzzle.

Drawing of the human figure: The evaluation displayed an evolutionary age of 3 years.

### Specialized Assessment of Autism

### Observational Scale for the Diagnosis of Autism ADOS 2

Selected module: Module 1—algorithm of some words
ADOS 2 is a semi-structured and standardized instrument to obtain information from the areas of: reciprocal social interaction and repetitive behavior, restricted to determine the clinical diagnosis of autism spectrum disorder (ASD). It includes various play-based activities such as: bubble play, releasing an inflated balloon, imitation activity, pretend birthday party, as well as free-play opportunities.

| EVALUATED AREAS | TOTAL |
|---|---|
| SOCIAL IMPACT | 7 |
| RESTRICTED BEHAVIOR | 1 |
| TOTAL | 8 |

### Equivalence for Diagnosis

| SCORE | EQUIVALENCE |
|---|---|
| LESS THAN 5 YEARS | |
| 12 OR MORE | AUTISM |
| 8-11 | AUTISM SPECTRUM |
| LESS THAN 7 | NO ASD |

The patient's mother was present during the application of the test. The patient managed to articulate single words and 3-word phrases with significant difficulty. She presented an exploratory and symbolic game. The patient mainly entertained herself by exploring the toys. She played representative games of caring for and feeding animals.

As can be seen in the results matrix, the patient obtained a score of 7 in the domain of social affectation (SA) and 1 in the domain of repetitive restricted behavior (RRB). Thus, from these results it can be inferred that the patient exhibited mild features that correspond to the autism spectrum.

The patient possessed adequate motor activity. Prior to starting the test application, the patient was shy, greeted people, collaborated with the proposed activities, and managed to maintain social interaction with difficulty. The patient often interacted mainly with her mother, to whom she frequently showed the toys. The patient managed to keep her attention for short periods (5-10 consecutive minutes) and played exploratory games. During short periods, shared enjoyment was achieved in social interaction activities such as bubbles. She focused her attention for brief periods on the proposed activities. Activities involving imitation were accomplished with difficulty, and eye contact was occasional.

The patient presented difficulty in imitation activities. The patient completed the anticipation activity of a routine with objects.

Regarding free play, the patient performed exploratory play. She represented baby and animal care; she played feeding them and reading them stories. The patient showed a special interest in the phone toy.

In response to name activities, the patient responded to the call of her name without difficulty. However, she struggled in activities requiring shared attention.

During the application of the tests, the patient did not have tantrums. The quality of the relationship with the evaluator was good. The patient presented recurring difficulties to ask for what she wanted. The patient showed sporadic immediate echolalia. She participated in the birthday party and collaborated without difficulty.

### Social Communication Questionnaire SCQ

| SCORE | CUT-OFF POINT FOR | |
|---|---|---|
| OBTAINED | DIAGNOSIS | DESCRIPTIVE CATEGORY |
| 15 | 15 | Presents mild features that are within the |
| | | spectrum. To a large extent, behaviors have |
| | | evolved favorably. |

The results obtained through the interview with the patient's mother allowed us to conclude that the patient presented features that are within the autism spectrum, at a mild degree. It is worth mentioning that the behavior associated with ASD showed a favorable evolution in relation to the mother's description.

The patient's behavior associated with ASD is described below:

| | | STEREOTYPICAL, |
|---|---|---|
| SOCIAL INTERACTION | COMMUNICATION | RESTRICTED AND |
| PROBLEMS | DIFFICULTIES | REPETITIVE BEHAVIOR |
| 1. Using other people's | 1. Difficulties in | 1. The repetitive use of objects |
| bodies to communicate has | conversation. | has greatly improved. |
| already been overcome. | 2. Stereotyped | 2. Weird sensory interests. |
| 2. Difficulty maintaining a | expressions. | 3. Hypersensitivity to certain |
| direct gaze, has improved | 3. Confusion of | sounds. |
| lately. | pronouns. | |
| 3. Difficulties making friends. | 4. Verbal routines. | |
| 4. Difficulties in social | | |
| interaction. | | |
| 5. Difficulties in fictional and | | |
| group games. | | |

### Development Evaluation

The Batelle Development Inventory was applied to the patient. This is an instrument for assessing developing skills and diagnosing possible deficiencies in different areas. The following results were obtained:

| Area | Subareas of age of development | | Age of development |
|---|---|---|---|
| Personal-Social | | | 39 months |
| Adaptive | | | 48 months |
| Motor | Gross Motor | 49 months | 42 months |
| | Fine Motor | 36 months | |
| Communication | Receptive | 46 months | 40 months |
| | Expressive | 36 months | |

| | |
|---|---|
| Cognitive | 43 months |
| Overall | 42 months |

Cognitive test: Patient was able to assemble a 6-piece puzzle in 1 minute. She matched circle, square, and triangle on a stringing board, skewered 9 figures out of 9 on a board, skewered 6 pegs on a board in 10 seconds, screwed and unscrewed jars, matched pictures, imitated actions, counted to 10, recognized numbers and sizes, classified by size and weight. In addition, she understood the concept of quantity but not the concept of “more” or the number-quantity relationship. She did not complete analogies.

Language test: The patient answered questions, although she repeated the question. She understood the use of objects and their functionality. The patient recognized simple negations, plural forms, and appropriately answered yes/no questions and elaborated 5-word sentences.

Motor test: The patient grabbed the pencil with an intermediate grip (tripod), copied a circle, vertical and horizontal strokes, made towers of 8 cubes, slightly screwed. She did not imitate square and triangle forms or V, T, X strokes. She was not able to cut with scissors following a line of 10 cm. She walked up and down the stairs alternating feet, balanced on one foot, kicked a ball, threw the ball with direction, ran, and jumped.

Personal/social test: In absence of her mother, the patient related to the examiner, asked for help, responded to orders, and followed instructions. The patient played next to a child, but she did not interact with him or other children. If they tried to greet her or shake hands, the patient hid behind the adult in charge.

Adaptive Testing: The patient paid attention, completed a task without supervision, stayed seated even when an adult was not present. She used a spoon to eat. She slightly removed her clothes and tried to put them on. Also, she was able to notify her need to go to the toilet, when needed.

### Conclusion

The diagnose was a global development delay with moderate affectation of fine psychomotor development and articulatory and comprehensive and expressive language.
The results suggested that the patient had a total IQ of 79 points, equivalent to low. There was a 95% chance that her total IQ is between 74-87.
The patient presented needs in verbal comprehension and processing speed. She displayed a significant strength in fluid reasoning.
The patient showed traits that are within the autism spectrum. Traits were in remission.
The patient had qualitative alterations in communication and social interaction.
Her psychomotor developmental age corresponded to 3 years and 0 months.

### Development Profile

Chronological age: 49 months (4 years and 1 month)
Age of global development: 42 months
Cognitive age: 43 months
Age in the adaptive area: 48 months
Global motor age: 42 months
Age-gross motor: 49 months
Fine motor age: 36 months
Age in the personal/social area: 39 months.
Mild delay in cognitive and personal/social development
Moderate delay in fine motor skills

### Evolution

The patient approved the first grade without academic problems and without any adaptation in the curriculum. She still had some communication problems, although she was able to make new friends on her own initiative.

### Case 5: Mixed Cerebral Palsy: Pyramidal and Extrapyramidal, Epilepsy, Constipation, Mental Retardation

Female patient of 4 years and 9 months of age. Daughter of non-consanguineous parents, born of a twin pregnancy by caesarean section at 30 weeks' gestation. The patient presented multiple complications in her neonatal period with prolonged mechanical ventilation, including neuroinfection and neonatal seizures.

At the time of the consultation, the patient did not present neck control, nor sitting position or language. Central hypotonia and peripheral hypertonia, dystonia and dyskinesia, ROT ++++/++, clonus presenting non-exhaustible, bilateral plantar flexor reflex was evidenced.

One year later, a simple brain tomography showed diffuse cortical-subcortical atrophy with areas of gliosis in the frontal region, greater ex-vacuo ventricular dilatation on the right. There were no advances regarding her neurological condition. Patient was administered with Baclofen to improve muscle tone and Levetiracetam to control seizures.

At eight years old, seizures were not controlled, and oxcarbazepine was added to the patient's treatment with Baclofen and Levetiracetam. Three and a half years later, the patient achieved incomplete neck control and peripheral tone decreased. Scoliosis, retractions, and joint deformities were visible. Dystonia and dyskinesia were persistent.

In the subsequent 7 and a half years, the patient presented epileptic seizures of irregular onset and with poor advances in her motor development. Administration of 27 mg of turmeric oil started and Baclofen was removed from the pharmacological treatment.

In the last 3 years, the patient attended medical controls irregularly, but she maintained her treatment with oxcarbazepine, lacosamide, clobazam, and turmeric oil. CBD and sacha inchi oil were also added to this treatment.

The patient's scoliosis did not progress in the last 3 years, and she achieved total neck control. She showed less hypertonia and a significant decrease in dystonia and dyskinesia.

She interacted appropriately with the environment. She was able to make eye contact and pronounce single, non-related words.

What is claimed is:

1. A method for treating neurological signs and symptoms in neurodegenerative diseases and developmental disorders comprising concomitantly administering to a subject in need thereof an effective amount of turmeric oil, sacha inchi oil, and cannabidiol.

2. The method of claim 1, wherein the administration of the oils and cannabidiol is oral.

3. The method of claim 1, wherein the neurological signs and symptoms include speech, language and communication disorders, social skills impairment, metacognition impairment, executive functions, and visual fixation patterns impairment, and pyramidal and extrapyramidal disorders associated with damage of the first or second motor neurons.
